(19) United States
(12) Patent Application Publication
(10) Pub. No.: US 2023/0330058 A1
(43) Pub. Date: Oct. 19, 2023

(54) SENOLYTIC COMPOUNDS AND COMPOSITIONS

(71) Applicant: NUCHIDO LIMITED, Tyne and Wear (GB)

(72) Inventors: Nichola Jane Conlon, Tyne and Wear (GB); Malcolm Philip Young, Tyne and Wear (GB)

(73) Assignee: NUCHIDO LIMITED, Tyne and Wear (GB)

(21) Appl. No.: 18/025,307

(22) PCT Filed: Sep. 8, 2021
(86) PCT No.: PCT/GB2021/052316

§ 371 Date: Mar. 8, 2023

(30) Foreign Application Priority Data
Sep. 9, 2020 (GB) 2014160.2

(51) Int. Cl. A61K 31/352; A61K 45/06; A61P 39/00; A61K 31/05

(52) U.S. Cl. CPC A61K 31/352; A61K 31/05; A61K 45/06; A61P 39/00

(57) ABSTRACT

There is described the use of an agent in the manufacture of a therapy for the treatment or alleviation of a senescence-associated disease, disorder or effect, wherein the agent is selected from one or more of A42548, A425619, alpha lipoic acid, apigenin, Artemisinin, BML-288, BTBHQ, EGCG, fluphenazine dihydrochloride, honokiol, LE300, magnolol, niclosamide, nicotinamide, nordihydroguaiaretic acid, quercetin, resveratrol and a mitochondrial uncoupler. There is also described the use of a mitochondrial uncoupler as an agent for the treatment of a senescence-associated disorder; and the use of an uncoupler in association with a senolytic agent.

### FIELD OF THE INVENTION

The present invention relates to compounds that exhibit selective senolytic activity, i.e. compounds that are capable of selectively killing senescent cells over non-senescent cells.

More particularly, the present invention relates to the use of selectively senolytic compounds for use in treating senescence-associated diseases or disorders, and compositions and methods relating thereto.

The invention also provides a method of mitigation of skin conditions and other effects of ageing using the selectively senolytic compounds and compositions of the invention.

### BACKGROUND TO THE INVENTION

Cells in the body are subject to a variety of oxidative, chemical and radiological insults during life. As the cells age, they acquire damage. In response to this damage, cells broadly follow one of three paths, they can:
endeavour to fix the damage;
undergo programmed cell death (“apoptosis”); or
undergo replicative senescence.

Fixing the damage can be thought of as the normal course of events: estimates suggest that there may be up to 60,000 DNA-damaging events per cell per day on average (“Epigenetic Reduction of DNA Repair in Progression to Cancer” Carol Bernstein & Harris Bernstein, Published: November 18^{th} 2015; DOI: 10.5772/60022), the effects of which are minimised by the active engagement of DNA repair mechanisms and other cellular maintenance and repair processes.

Apoptosis immediately removes the risk of tissue and organismal damage arising from malfunctioning damaged cells, although at the potential risk of compromising tissue structure if many cells terminate near-simultaneously.

Senescence may be an evolved mechanism for diminishing the risk from uncontrolled replication by malfunctioning cells that keeps the senescent cells in their tissue location for a longer time than would apoptosis.

One account of the chorus of inflammatory cytokines secreted by senescent cells (the “senescence associated secretory phenotype” or SASP) is that they are an invitation for immune clearance, evolved to steadily remove these cells at a rate manageable for maintaining tissue coherence. In any event, as age advances, recruitment of senescent cells steadily outstrips their removal, so that senescent cells represent a substantial minority of the cells in older tissues.

The continued production of SASP cytokines, the enhanced recruitment of further cells into senescence, and other deleterious consequences of the presence and activity of these populations of senescent cells underlie many aspects of ageing. An important set of results in the middle 2010s demonstrated that selective removal of senescent cells, and so removal of their malign influence, enabled substantial rejuvenation in multiple age-related biomarkers.

Accordingly, selective senolysis (and selective senostasis, in which senescent cells are dissuaded from their deleterious activities without killing them) have become major foci of research in therapeutic biogerontology, as well as in other areas in which senescent cells are thought to play a major role in specific disease pathogenesis (e.g. osteoarthritis, fibrosis, etc.).

Unfortunately, however, almost no senolytics are yet known that are simultaneously potent (i.e. deliverable at a feasible dose in Man), selective (i.e. disinclined to kill normal cells), and clean (i.e. without intolerable side effects).

Animal studies determined navitoclax to be a senolytic agent, inducing apoptosis in senescent cells, but less in non-senescent cells. Navitoclax and A-1331852 perhaps lead the field in potency, but these are or were originally intended as anti-cancer drugs attended by strong side-effect profiles, such as thrombocytopaenia, a condition characterised by abnormally low levels of platelets, in the blood, which reduces the ability of the blood to clot and is thus a bleeding diathesis.

It seems unlikely that side-effect profiles of this kind will realistically ever allow any of these molecules to be developed as a selective senolytic in an anti-ageing application. Such molecules would be taken mainly by people who are not suffering from cancer and are generally not in so desperate a condition as to make such side effects tolerable from a medical ethics perspective.

Hence, we set out to find potent, selective and clean senolytic agents that would be suitable for an anti-ageing application. In addition, we aimed for a potent, clean and selective senolytic that kills senescent cell-types quite generally; and is not limited to killing only a small number of senescent cell-types.

An interesting feature of disordered cells that have forked away from apoptosis and into replicative arrest is that they have typically passed several apoptotic checkpoints, but are held “above” apoptosis by a number of robust remaining checkpoints. A further interesting feature of the susceptibility of senescent cells to known senolytics is that they show strong cell-type effects. For example, a given senolytic molecule may kill senescent fibroblasts well, but may show negligible effects on senescent osteoblasts. These cell-type susceptibility effects are prima facie evidence that these different cell-types are not all held at a single common anti-apoptotic checkpoint, but that there are potentially many different mechanisms by which cells of different type are held in replicative senescence “above” apoptosis.

We have investigated small families of molecules and combinations of molecules that might persuade senescent cells past the last remaining checkpoints into apoptosis. This approach would not be expected to affect normal cells, since normal cells are not held above these checkpoints. Cancer cells of their many kinds search the space of available anti-apoptotic mechanisms in order to remain viable, and this space will include most or all of the mechanisms by which senescent cells are held “above” apoptosis, since cancer cells also have to find ways of avoiding their own apoptotic mechanisms. Hence, we were particularly interested in pro-apoptotic molecules from cancer research that kill a wide range of cancer cells at similar concentrations.

International patent application No. WO 2018/215795 (UKRI) describes agents, including cardiac aglycones or glycosides, such as, ouabain, digoxin, and K-strophanthin, for use in selectively killing one or more senescent cells.

### SUMMARY TO THE INVENTION

We have now found that certain compounds exhibit selective senolytic activity, i.e. are capable of selectively killing senescent cells over non-senescent cells.

Our experimental results showed that A42548, alpha lipoic acid, apigenin, Artemisinin, BML-288, BTBHQ, EGCG, fluphenazine dihydrochloride, honokiol, LE300, magnolol, niclosamide, nicotinamide, quercetin and resveratrol showed selective senolytic activity (exemplified in the Figures herein).

According to a first aspect of the invention there is provided the use of an agent in the manufacture of a therapy for the treatment or alleviation of a senescence-associated disease, disorder or effect, wherein the agent is selected from one or more of A42548, A425619, alpha lipoic acid, apigenin, Artemisinin, BML-288, BTBHQ, epigallocatechin gallate (EGCG), fluphenazine dihydrochloride, honokiol, LE300, magnolol, niclosamide, nicotinamide, nordihydroguaiaretic acid, quercetin, resveratrol and a mitochondrial uncoupler.

Reference to quercetin herein shall include derivatives of quercetin, such as, 3, 5, 7, 3′, 4′-pentahydroxyflavon, EMIQ isoquercitin, quercetin 3-O-glucoside, quercetin 3-O-rhamnoside; quercetin 3-O-rhamnozyl-(1→6)-glucoside (rutin); quercetin-3-O-beta-D-glucuronide and 3-methyl quercetin.

Reference to epigallocatechin gallate (EGCG) herein shall include green tea and/or green tea extract; and combinations thereof.

Reference to apigenin herein shall include parsley, parsley extract, chamomile and chamomile extract; and combinations thereof.

Reference to honokiol and/or magnolol herein shall include magnolia bark and magnolia bark extract; and combinations thereof.

Reference to resveratrol herein shall include red grape extract and polygonum cuspidatum root extract; and combinations thereof.

In a particular aspect of the invention there is provided the use of an agent in the manufacture of a therapy for the mitigation, alleviation or improvement of the senescence-associated effects of ageing, wherein the agent is selected from one or more of A42548, alpha lipoic acid, apigenin, Artemisinin, BML-288, BTBHQ, EGCG, fluphenazine dihydrochloride, honokiol, LE300, magnolol, niclosamide, nicotinamide, quercetin and resveratrol.

In a further aspect of the invention there is provided the use of an agent as herein described wherein the agent is selected from one or more of A42548, alpha lipoic acid, Artemisinin, BML-288, BTBHQ, fluphenazine dihydrochloride, honokiol, LE300, magnolol, niclosamide, nicotinamide, quercetin and resveratrol.

In one aspect of the invention the agent is A425619.

In one aspect of the invention the agent is A42548.

In another aspect of the invention the agent is alpha-lipoic acid (ALA).

In another aspect of the invention the agent is apigenin.

In another aspect of the invention the agent is artemisinin.

In another aspect of the invention the agent is BML288. BML288 is also known as romflumilast.

In another aspect of the invention the agent is BTBHQ. BTBHQ is also known as 2,5-di-tert-butylhydroquinone.

In another aspect of the invention the agent is fluphenazine dihydrochloride.

In another aspect of the invention the agent is honokiol.

In another aspect of the invention the agent is LE300. LE300 is 6,7,8,9,14,15-hexahydro-7-methyl-5H-indolo[3,2-f][3]benzazecine.

In another aspect of the invention the agent is magnolol.

In another aspect of the invention the agent is niclosamide. Niclosamide is selectively senolytic at concentrations that are already known to be reached safely in humans.

In another aspect of the invention the agent is niacinamide (also known as nicotinamide (NAM)).

In another aspect of the invention the agent is nordihydroguaiaretic acid.

In another aspect of the invention the agent is resveratrol. The resveratrol may comprise cis-resveratrol or trans-resveratrol. However, trans-resveratrol is preferred.

In another aspect of the invention the agent is a mitochondrial uncoupler. Mitochondrial uncouplers include Carbonyl cyanide-p-trifluoromethoxyphenylhydrazone (FCCP), Carbonyl cyanide m-chlorophenyl hydrazone (CCCP), N5,N6-bis(2-Fluorophenyl)[1,2,5]oxadiazolo[3,4-b]pyrazine-5,6-diamine (BAM 15) and 2,4-Dinitrophenol (DNP).

In another aspect of the invention the agent comprises a combination of two or more of A42548, A425619, alpha lipoic acid, apigenin, Artemisinin, BML-288, BTBHQ, EGCG, fluphenazine dihydrochloride, honokiol, LE300, magnolol, niclosamide, nicotinamide, nitazoxanide, nordihydroguaiaretic acid, quercetin and resveratrol.

In a further aspect of the invention the agent comprises a combination of two or more of A42548, alpha lipoic acid, apigenin, Artemisinin, BML-288, BTBHQ, EGCG, fluphenazine dihydrochloride, honokiol, LE300, magnolol, niclosamide, nicotinamide, nitazoxanide, quercetin and resveratrol.

In one aspect of the invention the agent is a combination comprising resveratrol, alpha lipoic acid, apigenin, nicotinamide, quercetin and EGCG.

In one aspect of the invention the agent is a combination comprising resveratrol, alpha lipoic acid, nicotinamide, quercetin and EGCG.

In another aspect of the invention the agent is a combination comprising resveratrol and EGCG.

In another aspect of the invention the agent is a combination comprising magnolol and honokiol.

In another aspect of the invention the agent is a combination comprising honokiol, magnolol and niclosamide.

In another aspect of the invention the agent is a combination comprising honokiol, magnolol and nitazoxanide.

In another aspect of the invention the agent is a combination comprising A42548, artemisinin, BNIL-288, BTBHQ, honokiol, LE300, magnolol and niclosamide.

According to a further aspect of the invention there is provided the use of a mitochondrial uncoupler as an agent in the manufacture of a therapy for the treatment or alleviation of a senescence-associated disease, disorder or effect.

In the use according to this aspect of the invention the mitochondrial uncoupler may be selected from one or more of Carbonyl cyanide-p-trifluoromethoxyphenylhydrazone (FCCP), Carbonyl cyanide m-chlorophenyl hydrazone (CCCP), N5,N6-bis(2-Fluorophenyl)[1,2,5]oxadiazolo[3,4-b]pyrazine-5,6-diamine (BAM 15) and 2,4-Dinitrophenol (DNP); and combinations thereof.

According to a further aspect of the invention there is provided a composition comprising an amount of an agent effective for the treatment or alleviation of a senescence-associated disease, disorder or effect, wherein the agent is selected from one or more of A42548, A425619, alpha lipoic acid, apigenin, Artemisinin, BML-288, BTBHQ, EGCG, fluphenazine dihydrochloride, honokiol, LE300, magnolol, niclosamide, nicotinamide, nordihydroguaiaretic acid, quercetin and resveratrol.

According to a further aspect of the invention there is provided a composition comprising an amount of an agent effective for mitigation, alleviation or improvement of the senescence-associated effects of ageing, wherein the agent is selected from one or more of A42548, alpha lipoic acid, apigenin, Artemisinin, BML-288, BTBHQ, EGCG, fluphenazine dihydrochloride, honokiol, LE300, magnolol, niclosamide, nicotinamide, quercetin and resveratrol.

According to a further aspect of the invention there is provided a composition comprising an amount of an agent effective for mitigation, alleviation or improvement of the senescence-associated effects of ageing, wherein the agent is selected from one or more of A42548, alpha lipoic acid, Artemisinin, BNIL-288, BTBHQ, fluphenazine dihydrochloride, honokiol, LE300, magnolol, niclosamide, nicotinamide, quercetin and resveratrol.

In one aspect of the invention the composition comprises A425619.

In one aspect of the invention the composition comprises A42548.

In another aspect of the invention the composition comprises alpha-lipoic acid (ALA).

In another aspect of the invention the composition comprises apigenin.

In another aspect of the invention the composition comprises artemisinin.

In another aspect of the invention the composition comprises BML288.

In another aspect of the invention the composition comprises BTBHQ.

In another aspect of the invention the composition comprises fluphenazine dihydrochloride.

In another aspect of the invention the composition comprises honokiol.

In another aspect of the invention the composition comprises LE300.

In another aspect of the invention the composition comprises magnolol.

In another aspect of the invention the composition comprises niclosamide.

In another aspect of the invention the composition comprises niacinamide.

In another aspect of the invention the composition comprises nordihydroguaiaretic acid.

In another aspect of the invention the composition comprises resveratrol.

The amount of active agents may vary. However, illustrative, non-limiting, daily amounts may include, but shall not be limited to, resveratrol, about 20 µM; alpha-lipoic acid, about 40 µM; apigenin, about 1 µM; niacinamide, about 40 µM; quercetin, about, 5 µM; epigallocatechin gallate, about 40 µM; niclosamide, about 1 µM; fluphenazine dihydrochloride, about 10 µM; magnolol, about 10 µM; honokiol, about 10 µM; BTBHQ, about 6.25 µM; BML288, about 5 µM; artemisinin, about 5 µM; nordihydroguaiaretic acid, about 10 µM; LE300, about 10 µM; and A425619, about 10 µM.

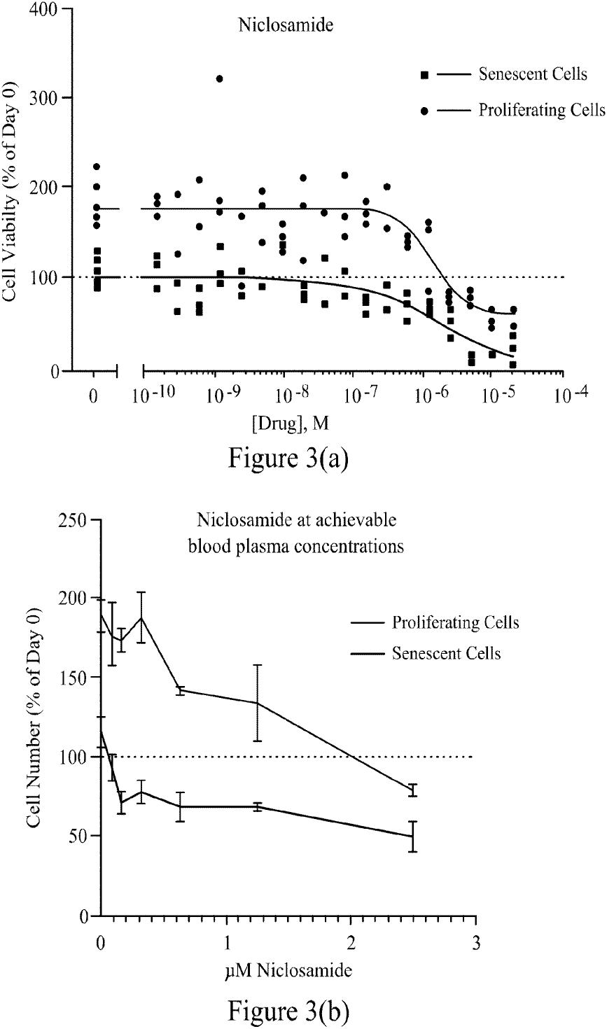

Of the agents investigated, niclosamide showed particular promise, since it was selectively senolytic at concentrations that are already known to be reached safely in humans (FIG. 3). Thus, in another aspect of the invention the composition comprises niclosamide.

In another aspect of the invention the composition comprises a combination of two or more of A42548, A425619, alpha lipoic acid, apigenin, Artemisinin, BML-288, BTBHQ, EGCG, fluphenazine dihydrochloride, honokiol, LE300, magnolol, niclosamide, nicotinamide, nitazoxanide, nordihydroguaiaretic acid, quercetin and resveratrol.

In another aspect of the invention the composition comprises a combination of two or more of A42548, alpha lipoic acid, apigenin, Artemisinin, BML-288, BTBHQ, EGCG, fluphenazine dihydrochloride, honokiol, LE300, magnolol, niclosamide, nicotinamide, nitazoxanide, quercetin and resveratrol.

In one aspect of the invention the composition comprises a combination comprising resveratrol, alpha lipoic acid, apigenin, nicotinamide, quercetin and EGCG.

In one aspect of the invention the composition comprises a combination comprising resveratrol, alpha lipoic acid, nicotinamide, quercetin and EGCG.

In another aspect of the invention the composition comprises a combination comprising resveratrol and EGCG.

In another aspect of the invention the composition comprises a combination comprising magnolol and honokiol.

In another aspect of the invention the composition comprises a combination comprising honokiol, magnolol and niclosamide.

In another aspect of the invention the agent is a combination comprising honokiol, magnolol and nitazoxanide.

In another aspect of the invention the composition comprises a combination comprising A42548, artemisinin, BNIL-288, BTBHQ, honokiol, LE300, magnolol and niclosamide.

We examined combinations of agents in assays of selective senolysis using human fibroblasts. Resveratrol-quercetin-EGCG was much better than quercetin alone. Thus, according to a further aspect of the invention the composition comprises a combination of resveratrol, EGCG and quercetin.

The combination of agents identified as ABFGH showed selectivity comparable to the best known senolytic agents, but involved only agents and concentrations that are known to be safe through many millions of patient exposures. Thus, according to a further aspect of the invention the composition comprises the combination ABFGH. For the avoidance of doubt, A is resveratrol; B is alpha-lipoic acid; F is niacinamide; G is quercetin; and H is epigallocatechin gallate.

According to a further aspect of the invention the composition comprises niclosamide in combination with one or more of resveratrol, alpha-lipoic acid (ALA), niacinamide (also known as nicotinamide (NAM)), epigallocatechin gallate (EGCG) and fluphenazine dihydrochloride.

Apoptosis regulator BAX, also known as bcl-2-like protein 4, is a protein that in humans is encoded by the BAX gene. BAX is a member of the BCL-2-gene family. BCL2 family members form hetero- or homodimers and act as anti- or pro-apoptotic regulators that are involved in a wide variety of cellular activities. A small number of molecules from the family of pro-apoptotics that inhibit BCL2/X are among the most potent senolytics currently known. One example of a pro-apoptotic that inhibits BCL2/X is navitoclax. Another example is A1331852, which is also a potent and selective BCL-XL-selective inhibitor. A1331852 is 3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid.

As also noted, though, at concentrations likely to be effective they are each attended by highly undesirable side-effects that would severely restrict their suitability as senolytics.

However, we have surprisingly found that the use of a mitochondrial uncoupler in association with a senolytic agent may reduce undesirable side-effects of the senolytic agent; or the mitochondrial uncoupler may increase the potency of the senolytic agent, thus enabling a lower dose, or sub-therapeutic dose, of the senolytic agent to be administered, thus achieving a similar therapeutic effect, but with reduced side effects.

Therefore, according to this aspect of the invention there is provided the use of a mitochondrial uncoupler in association with a senolytic agent in the manufacture of a therapy for the treatment or alleviation of a senescence-associated disease, disorder or effect.

When the mitochondrial uncoupler is used in association with a sub-therapeutic dose of a senolytic agent, the term “sub-therapeutic dose” should be understood to mean a dose that would be below the conventionally known therapeutic threshold for the treatment or alleviation of a senescence-associated disease, disorder or effect. In the context of the present invention the sub-therapeutic dose may comprise a dose that is 10% w/w lower than the conventionally administered dose; or 20% w/w lower than the conventionally administered dose; or 30% w/w lower than the conventionally administered dose; or 40% w/w lower than the conventionally administered dose; or 50% w/w lower than the conventionally administered dose.

In the use according to this aspect of the invention the mitochondrial uncoupler may be selected from one or more of Carbonyl cyanide-p-trifluoromethoxyphenylhydrazone (FCCP), Carbonyl cyanide m-chlorophenyl hydrazone (CCCP), N5,N6-bis(2-Fluorophenyl)[1,2,5]oxadiazolo[3,4-b]pyrazine-5,6-diamine (BAM 15) and 2,4-Dinitrophenol (DNP); and combinations thereof.

Thus, according to a further aspect of the invention there is provided a composition as herein described which includes a BCL2/X inhibitor, such as, one or more of navitoclax and A1331852.

Mitochondrial uncoupling is a dissociation between mitochondrial membrane potential generation and its use for mitochondria-dependent ATP synthesis. An uncoupler or uncoupling agent is a molecule that disrupts oxidative phosphorylation in mitochondria by dissociating the reactions of ATP synthesis from the electron transport chain. The result is that the cell or mitochondrion expends energy to generate a proton motive force, but the proton motive force is dissipated before the ATP synthase can recapture this energy and use it to make ATP. Uncouplers are capable of transporting protons through mitochondrial and lipid membranes.

More specifically, there is provided a composition as herein described which includes one or more of Carbonyl cyanide-p-trifluoromethoxyphenylhydrazone (FCCP), Carbonyl cyanide m-chlorophenyl hydrazone (CCCP), N5,N6-bis(2-Fluorophenyl)[1,2,5]oxadiazolo[3,4-b]pyrazine-5,6-diamine (BAM 15) and 2,4-Dinitrophenol (DNP).

Metformin is an FDA-approved first-line drug for the treatment of type 2 diabetes, used successfully for over 60 years with an outstanding safety record. Studies in the biology of aging have shown that metformin can delay aging in animals. These findings point to the likelihood that metformin may influence fundamental aging factors that underlie multiple age-related conditions in humans.

Surprisingly, we find that metformin is a senolytic at physiological doses if glycolysis is suppressed.

Thus, according to a further aspect of the invention there is provided a composition as herein described which includes metformin in combination with any of the aforementioned senolytic compounds. We have surprisingly found that metformin is senolytic when glycolysis is suppressed. Therefore, advantageously the composition of the invention may include metformin with one or more antiglycolytic agents or antiglycolytic precursors. The term “antiglycolytic” is used broadly herein to include any substance that at least retards glucose consumption of living cells.

Examples of antiglycolytic agents include, but shall not be limited to, fluorides, glyceraldehydes, mannose, glucosamine, mannoheptulose, sorbose-6-phophate, trehalose-6-phosphate, maleimide, oleanolic acid, iodoacetates, and the like, and combinations thereof. Examples of antiglycolytic agent precursors include, but are not limited to, enzymes, e.g., trehalose-6-phosphate synthase, and the like. For example, the antiglycolytic agent may be glyceraldehydes, e.g., D-glyceraldehyde, L-glyceraldehyde, or a racemic mixture of D- and L-glyceraldehyde. As noted above, fluorides may be used, e.g., sodium fluoride, potassium fluoride, etc.

Additional active agents may also be included in the composition of the invention. Such additional active agents shall include but shall not be limited to, nitazoxanide and myricetrin.

A particular composition of the invention comprising nitazoxanide and myricetrin is a composition comprising honokiol, magnolol, myricetrin and nitazoxanide.

We have surprisingly found that a composition comprising a combination of magnolol and honokiol may be especially advantageous on that, inter alia, the combination has a synergistic effect on other senolytic compounds and is able to increase the potency of other senolytics.

According to a further aspect of the invention there is provided a composition comprising an amount of an agent effective for the treatment or alleviation of a senescence-associated disease, disorder or effect, wherein the agent is a mitochondrial uncoupler.

According to a further aspect of the invention there is provided a composition comprising a mitochondrial uncoupler in association with an amount of an agent effective for the treatment or alleviation of a senescence-associated disease, disorder or effect.

In a composition according to these aspects of the invention the mitochondrial uncoupler may be selected from one or more of Carbonyl cyanide-p-trifluoromethoxyphenylhydrazone (FCCP), Carbonyl cyanide m-chlorophenyl hydrazone (CCCP), N5,N6-bis(2-Fluorophenyl)[1,2,5]oxadiazolo[3,4-b]pyrazine-5,6-diamine (BAM 15) and 2,4-Dinitrophenol (DNP); and combinations thereof; and combinations thereof.

The composition of the invention will generally include an acceptable excipient.

The compositions of the invention may also include one or more bioavailability enhancers or skin penetration enhancers. Such bioavailability enhancers or skin penetration enhancers shall include, but shall not be limited to, DMSO, decyl methyl sulfoxide, N-dodecyl pyrrolidone, decanol, dodecanol, an organic acid such as oleic acid, zinc, vitamin C and piperine (Bioperine®) or the like; and combinations thereof. In one aspect of the invention the bioavailability enhancers or skin penetration enhancers include zinc, vitamin C and piperine (Bioperine®); and combinations thereof.

The senescence-associated effects of ageing may include, but shall not be limited to, Chronic Obstructive Pulmonary Disease (COPD), oesteoarthritis, sarcopenia, cachexia, osteoporosis, type 2 diabetes, atherosclerosis, idiopathic pulmonary fibrosis, glaucoma, liver cirrhosis (Ref: McHug & Gil, 2018: https://www.nebi.nlm.nih.gov/pmc/articles/PMC5748990/) and skin conditions. When the senescence-associated effects of ageing is a skin condition, it may include age-related skin conditions, skin conditions related to sun exposure, skin conditions related to pollution exposure, skin conditions related to oxidative stress, and skin conditions related to lifestyle choices, such as diet, alcohol and/or smoking. In addition, the compositions of the invention may be advantageous in the mitigation, alleviation or improvement of skin conditions related to inflammatory skin disorders and skin conditions related autoimmune disease skin disorders. The compositions of the invention may be advantageous in the mitigation, alleviation or improvement of other age-related conditions, such as, but not limited to, increased frailty, loss of resilience, loss of muscle strength, loss of muscle endurance, loss of energy, loss of cognitive sharpness, loss of memory, etc. More specifically, the compositions of the invention may be advantageous in the mitigation, alleviation or improvement of other age-related conditions, such as, but not limited to, atherosclerosis and cardiovascular disease, cancer, arthritis, cataracts, osteoporosis, type 2 diabetes, hypertension, Alzheimer’s disease, glomerulosclerosis/decline in renal function; the incidence of which increases with aging.

Age-related skin conditions that may be mitigated, alleviated or improved, shall include, but shall not be limited to, one or more of sagging, wrinkles, skin elasticity, skin ageing, skin moisture, wounds, acne, skin darkening, skin whitening, pigmentation, age-spots, loss of radiance, puffiness, uneven skin tone, redness, rosacea, loss of barrier function, loss of skin resilience, loss of firmness, stretch-marks, cellulite and dryness.

Skin conditions related to sun exposure that may be mitigated, alleviated or improved, include, but shall not be limited to, one or more of actinic keratoses, freckles, lentigines or age spots, moles, photosensitivity, polymorphous light eruption, seborrheic keratoses, skin cancer (such as melanoma, squamous cell carcinoma, basal cell carcinoma), solar elastosis or wrinkles and sun burn.

Skin conditions related to inflammatory skin disorders that may be mitigated, alleviated or improved, include, but shall not limited to, one or more of acne, asteatotic eczema, atopic dermatitis, contact dermatitis, discoid eczema, eczematous drug eruptions, erythema multiforme, erythroderma, gravitational/varicose eczema, hand eczema, keratosis lichenoides chronica, lichen nitidus, lichen planus, lichen simplex, lichen striatus, mycosis fungoides, pityriasis lichenoides, psoriasis, seborrheic dermatitis, Stevens-Johnson Syndrome, toxic epidermal necrolysis and vasculitis.

Skin conditions related autoimmune disease skin disorders that may be mitigated, alleviated or improved, include, but shall not limited to, one or more of alopecia areata, bullous pemphigoid, dermatomyositis, dystrophic epidermolysis bullosa, eosinophilic fasciitis, pemphigus vulgaris, psoriasis, pyoderma gangrenosum, scleroderma, systemic lupus erythematosus and vitiligo.

The composition of the present invention may be administered topically, orally or parenterally; or may comprise controlled, modified or extended release formulations comprising suitable mitigation amounts of the desired active components in the form of powders, granules, sterile parenteral solutions or suspensions, oral solutions or suspensions, oil water emulsions as well as implants and microencapsulated delivery systems.

### Parenteral Administration

Thus, according to one aspect of the invention there is provided the composition as herein described for parenteral administration.

When the composition of the invention is administered parenterally, it may be in the form of an intramuscular, intravenous, subcutaneous, intraperitoneal, local or transdermal bolus injection or continuous infusion.

### Topical Administration

Preferably, the composition of the invention may be administered topically or transdermally. Thus, according to this aspect of the invention there is provided the composition as herein described for topical administration. According to a further aspect of the invention there is provided the composition as herein described for transdermal administration.

Suitable formulations for topical or transdermal application include an effective amount of the composition of the invention comprise the active components as herein defined with one or more carriers. Carriers include absorbable pharmacologically acceptable solvents to assist passage into the skin of the host.

Suitable formulations for topical application, e.g., to the skin and eyes, include aqueous solutions, suspensions, ointments, creams, gels, sprayable formulations, transdermal patch or bandage e.g., for delivery by aerosol or the like. Such topical delivery systems will in particular be appropriate for dermal application, for prophylactic use in sun creams, lotions, sprays and the like. They are thus particularly suited for use in topical, including cosmetic, formulations well-known in the art. Such formulations may contain solubilisers, stabilizers, tonicity enhancing agents, buffers and preservatives.

Transdermal devices may be in the form of a bandage comprising a backing member, a reservoir containing the composition of the invention optionally with carriers, optionally a rate controlling barrier to deliver the composition of the invention to the skin of the host at a controlled and predetermined rate over a prolonged period of time, and means to secure the device to the skin.

### Oral Administration

Thus, according to one aspect of the invention there is provided the composition as herein described for oral administration.

When the composition of the invention is administered orally, it may be in the form of tablets or capsules.

The compositions of the invention can be made up in a solid form including capsules, tablets, pills, granules, powders, food bar or confectionery; or in a liquid form including solutions, suspensions or emulsions or in the form of a syrup, linctus, elixir, a liquid beverage, such as a yoghurt drink, or a foodstuff, such as a yoghurt.

The compositions can be subjected to conventional operations such as sterilization and/or can contain conventional inert diluents, lubricating agents, or buffering agents, as well as adjuvants, such as preservatives, stabilizers, wetting agents, emulsifiers and buffers, etc.

Typically, when the compositions are in the form of tablets or capsules, e.g. gelatin capsules, the compositions may comprise the active components together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also;
c) binders, e.g., magnesium aluminium silicate, starch paste, gelatin, tragacanth, methylcellulose, sodium carboxymethylcellulose and/or polyvinylpyrrolidone; if desired;
d) disintegrants, e.g., starches, agar, alginic acid or its sodium salt, or effervescent mixtures; and/or
e) absorbents, colourants, flavours and sweeteners.

Tablets may be either film coated or enteric coated according to methods known in the art.

Suitable compositions for oral administration include an effective amount of the active components described herein in the form of tablets, lozenges, aqueous or oily suspensions, dispersible powders or granules, food bar, confectionery, solution, emulsion, hard or soft capsules, a syrup, linctus, elixir, a liquid beverage or a foodstuff.

Compositions intended for oral use can be prepared according to any method known in the art for the manufacture of effective compositions; and such compositions can contain one or more additional agents selected from the group consisting of sweetening agents, flavouring agents, colouring agents and preserving agents in order to provide elegant and palatable preparations.

Tablets contain the composition comprising the active components herein described, in admixture with non-toxic orally acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets may be uncoated or coated by known techniques to delay disintegration and absorption in the gastrointestinal tract and thereby provide a sustained action over a longer period. For example, a time delay material such as glyceryl monostearate or glyceryl distearate can be employed.

Formulations for oral use can be presented as hard gelatin capsules wherein the composition comprising the active components is mixed with an inert solid diluent, for example, calcium carbonate, calcium phosphate or kaolin, or as soft gelatin capsules wherein the composition comprising the active components is mixed with water or an oil medium, for example, peanut oil, liquid paraffin or olive oil.

The soft capsule can be prepared using techniques well known in the art. For example, soft capsules are typically produced using a rotary die encapsulation process. Active agent formulations are fed into the encapsulation machine by gravity. In an embodiment, the formulation comprises pharmaceutical excipients such as olive oil, gelatin, glycerin, purified water, beeswax yellow, sunflower lecithin, silicon dioxide, titanium dioxide, F. D. & C Blue 1 and F. D. & C Red 4, microcrystalline cellulose, hypromellose, vegetable magnesium stearate, and/or silica.

A capsule shell can comprise one or more plasticizers such as glycerin, sorbitol, sorbitans, maltitol, glycerol, polyethylene glycol, polyalcohols with 3 to 6 carbon atoms, citric acid, citric acid esters, triethyl citrate and combinations thereof. In an embodiment, the plasticizer is glycerin.

In addition to the plasticizer(s), the capsule shell can include other suitable shell additives such as opacifiers, colourants, humectants, preservatives, flavourings, and buffering salts and acids.

Opacifiers are used to opacify the capsule shell when the encapsulated active agents are light sensitive. Suitable opacifiers include, but not limited to, titanium dioxide, zinc oxide, calcium carbonate and combinations thereof. In an embodiment, the opacifier is titanium dioxide.

Colourants can be used to for marketing and product identification and/or differentiation purposes. Suitable colourants include synthetic and natural dyes and combinations thereof.

Humectants can be used to suppress the water activity of the softgel. Suitable humectants include glycerin and sorbitol, which are often components of the plasticizer composition. Due to the low water activity of dried, properly stored softgels, the greatest risk from microorganisms comes from molds and yeasts. For this reason, preservatives can be incorporated into the capsule shell. Suitable preservatives include alkyl esters of p-hydroxy benzoic acid such as methyl, ethyl, propyl, butyl and heptyl (collectively known as “parabens”) or combinations thereof.

According to a further aspect of the invention there is provided a method of mitigation, alleviation or improvement of the effects of ageing in a host, said method comprising the administration of an effective amount of a composition comprising one or more of A42548, A425619, alpha lipoic acid, apigenin, Artemisinin, BML-288, BTBHQ, EGCG, fluphenazine dihydrochloride, honokiol, LE300, magnolol, niclosamide, nicotinamide, nordihydroguaiaretic acid, quercetin, resveratrol and a mitochondrial uncoupler, as herein described.

According to a further aspect of the invention there is provided a method of mitigation, alleviation or improvement of the effects of ageing in a host, said method comprising the administration of an effective amount of a composition comprising one or more of A42548, alpha lipoic acid, apigenin, Artemisinin, BML-288, BTBHQ, EGCG, fluphenazine dihydrochloride, honokiol, LE300, magnolol, niclosamide, nicotinamide, quercetin, resveratrol and a mitochondrial uncoupler.

According to a further aspect of the invention there is provided a method of mitigation, alleviation or improvement of the effects of ageing in a host, said method comprising the administration of an effective amount of a composition comprising one or more of A42548, alpha lipoic acid, Artemisinin, BNIL-288, BTBHQ, fluphenazine dihydrochloride, honokiol, LE300, magnolol, niclosamide, nicotinamide, quercetin, resveratrol and a mitochondrial uncoupler.

According to this aspect of the invention the method of mitigation, alleviation or improvement of the senescence-associated effects of ageing may comprise the administration of an effective amount of a senolytic agent as herein described or a combination of two or more senolytic agents as herein described.

According to a further aspect of the invention there is provided a method of mitigation, alleviation or improvement of the senescence-associated effects of ageing; said method comprising the administration of an effective amount of a mitochondrial uncoupler.

According to a further aspect of the invention there is provided a method of mitigation, alleviation or improvement of the senescence-associated effects of ageing; said method comprising the administration of an effective amount of a mitochondrial uncoupler in association with an amount of an agent effective for the treatment or alleviation of a senescence-associated disease, disorder or effect.

According to these aspects of the invention the mitochondrial uncoupler may be selected from one or more of Carbonyl cyanide-p-trifluoromethoxyphenylhydrazone (FCCP), Carbonyl cyanide m-chlorophenyl hydrazone (CCCP), N5,N6-bis(2-Fluorophenyl)[1,2,5]oxadiazolo[3,4-b]pyrazine-5,6-diamine (BAM 15) and 2,4-Dinitrophenol (DNP); and combinations thereof.

The method according to this aspect of the invention may comprise the mitigation, alleviation or improvement of age related skin conditions, skin conditions related to sun exposure, skin conditions related to pollution exposure, skin conditions related to oxidative stress, and skin conditions related to lifestyle choices, such as diet, alcohol and/or smoking. In addition, the method of the invention may be advantageous in the mitigation, alleviation or improvement of skin conditions related to inflammatory skin disorders and skin conditions related autoimmune disease skin disorders.

Selection of a particular effective dose can be determined (e.g., via clinical trials) by a person skilled in the art based upon the consideration of several factors which will be known to the person skilled in the art, such as, the skin disorder to be mitigated, alleviated or improved; the nature and severity of the skin disorder being treated, the body mass of the host; and the like. The precise dose employed in the mitigation, alleviation or improvement of the skin disorder may also depend upon the route of administration. Effective doses can be extrapolated from dose-response curves derived from in vitro or animal model test systems.

However, in general, satisfactory results may be obtained at a daily dosage of the composition of the invention of from about 0.1 to about 500 mg/kg body weight; or from about 1 to about 400 mg/kg; or from about 1 to about 300 mg/kg; or from about 1 to about 200 mg/kg; or from about 1 to about 100 mg/kg; or from about 10 to about 50 mg/kg; administered, for example, in divided doses up to three or four times a day, e.g. twice daily, or in sustained release form.

It is often practical to administer the daily dose of the composition of the invention at various hours of the day. The amount of the active composition administered may depend on such factors as the solubility of the active composition, the formulation used, subject condition (such as weight), and/or the route of administration.

The present invention will now be described by way of example only and with reference to the accompanying figures, in which:

FIG. 1 illustrates the selective senolytic activity of the combination of drugs A, B, C, F, G and H (A-resveratrol (40 µM); B-alpha lipoic acid (10 µM); C-apigenin (1 µM); F-nicotinamide (40 µM); G-Quercetin (1 µM); and H-EGCG (40 µM));

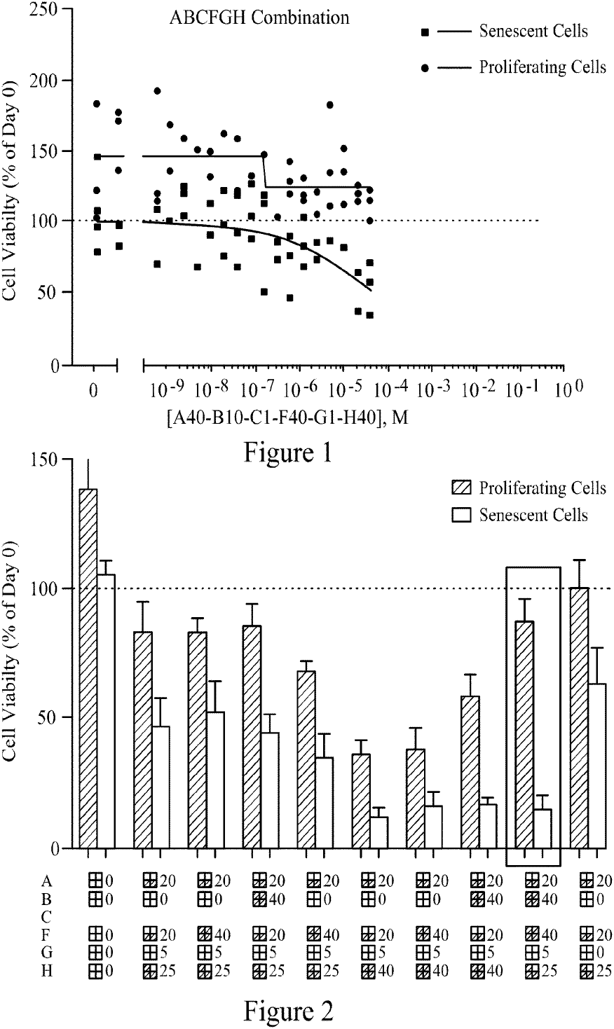

FIG. 2 illustrates the selective senolytic activity of the combination of drugs A, B, F, G and H (A-Resveratrol (20 µM); B-Alpha lipoic acid (40 µM); F-Nicotinamide (40 µM); G-Quercetin (5 µM); and H-EGCG (40 µM));

FIG. 3(a) illustrates the selective senolytic activity of niclosamide;

FIG. 3(b) illustrates the selective senolytic activity of niclosamide at achievable blood plasma concentrations;

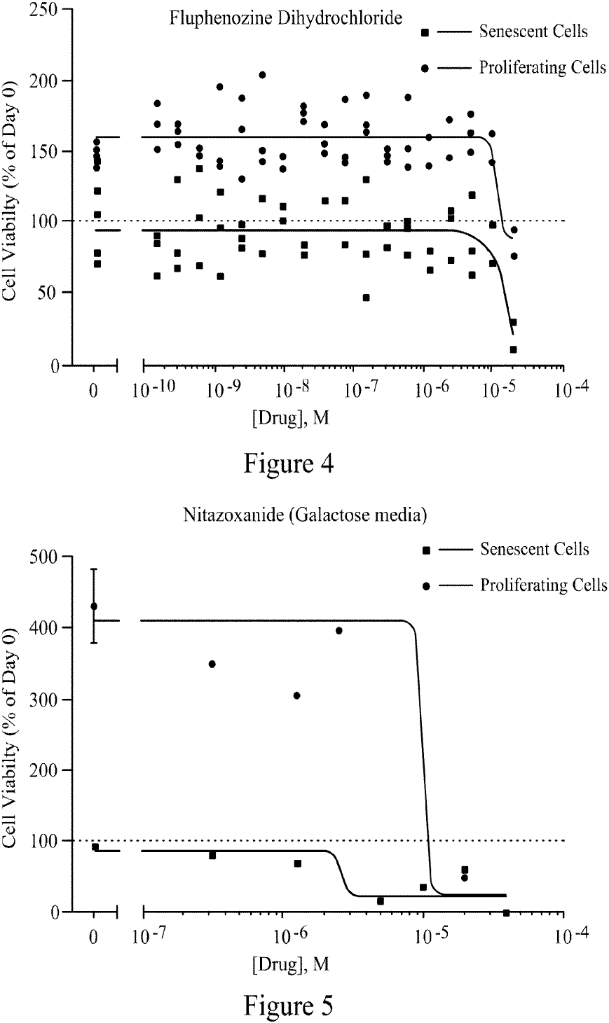

FIG. 4 illustrates the selective senolytic activity of fluphenazine hydrochloride;

FIG. 5 illustrates the selective senolytic activity of nitazoxanide in galactose media;

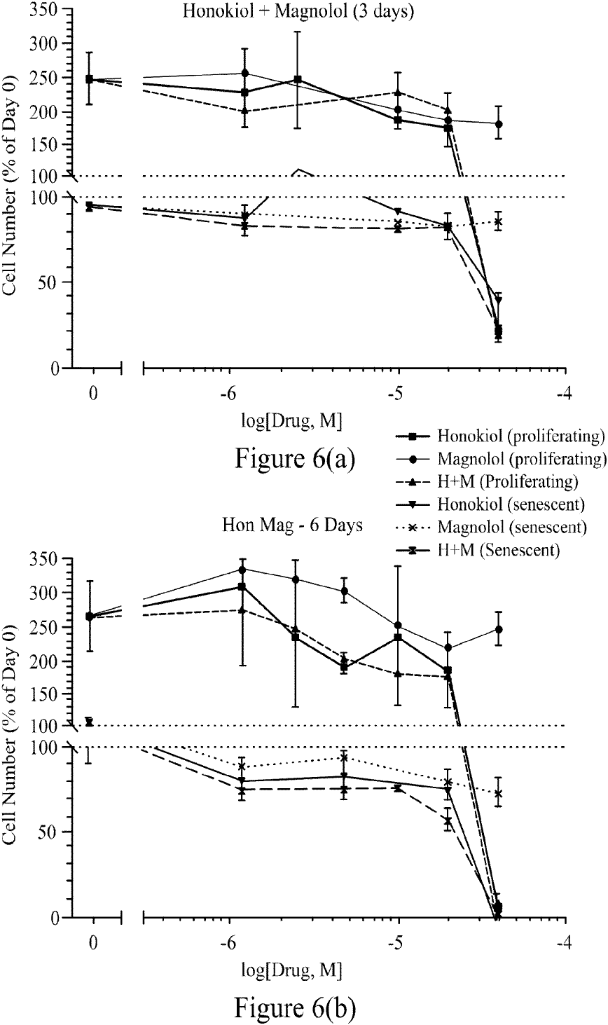

FIGS. 6(a) and (b) illustrate the selective senolytic activity of the combination of honokiol and magnolol after 3 days and 6 days respectively;

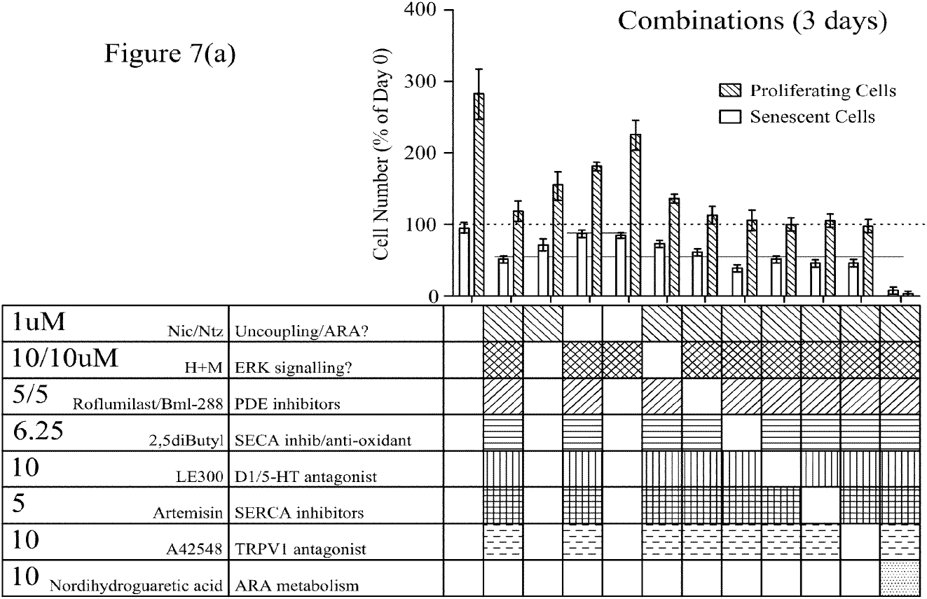

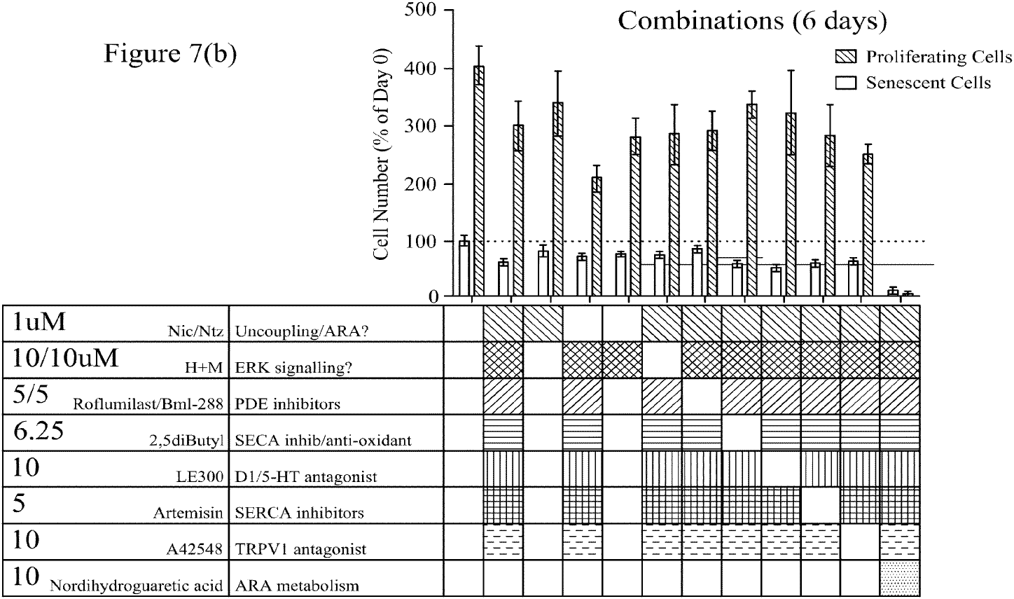

FIG. 7(a) illustrates the selective senolytic activity of the combination of niclosamide, honokiol, magnolol, BML-288, BTBHQ, LE300, artemisin and A42548;

FIG. 7(b) illustrates the selective senolytic activity of the combination of nitazoxanide, honokiol, magnolol, BML-288, BTBHQ, LE300, artemisin and A42548;

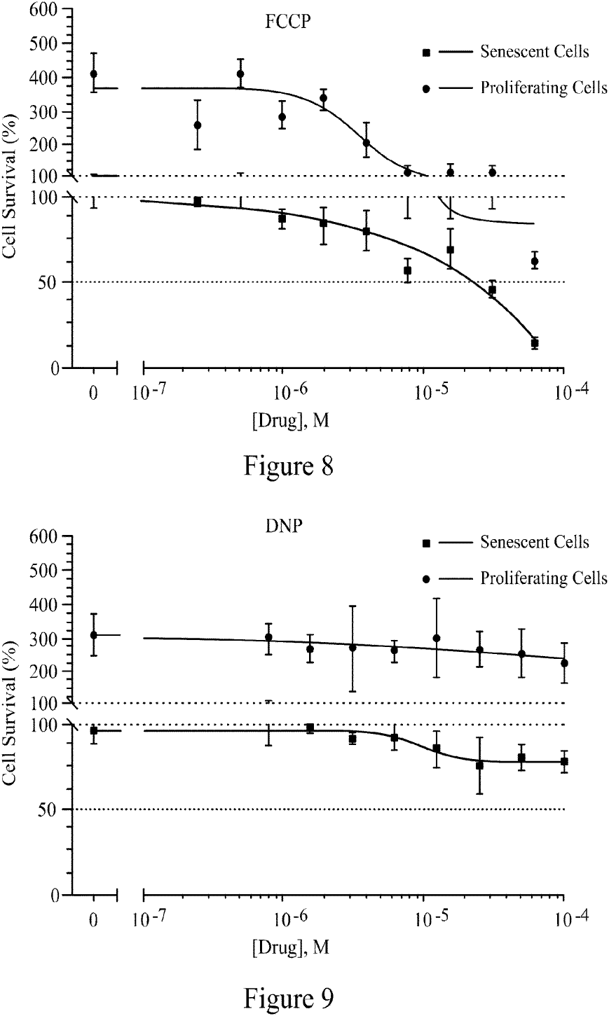

FIG. 8 illustrates the selective senolytic activity of FCCP;

FIG. 9 illustrates the selective senolytic activity of DNP;

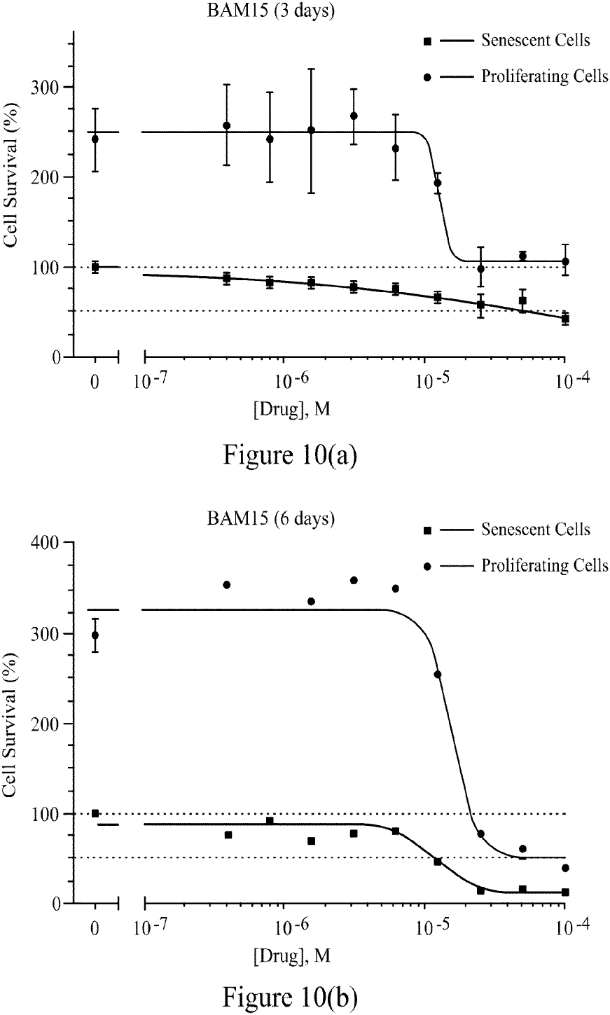

FIGS. 10(a) and (b) illustrate the selective senolytic activity of BAM15 treatment over three and six days respectively;

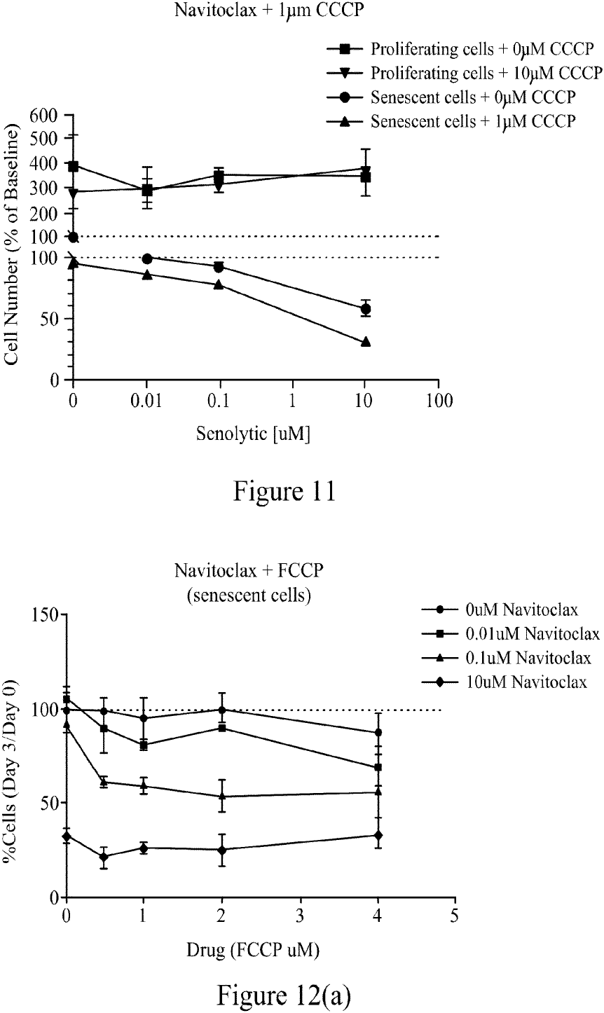

FIG. 11 illustrates that CCCP increases the senolytic effect of navitoclax, increasing efficacy at lower concentrations;

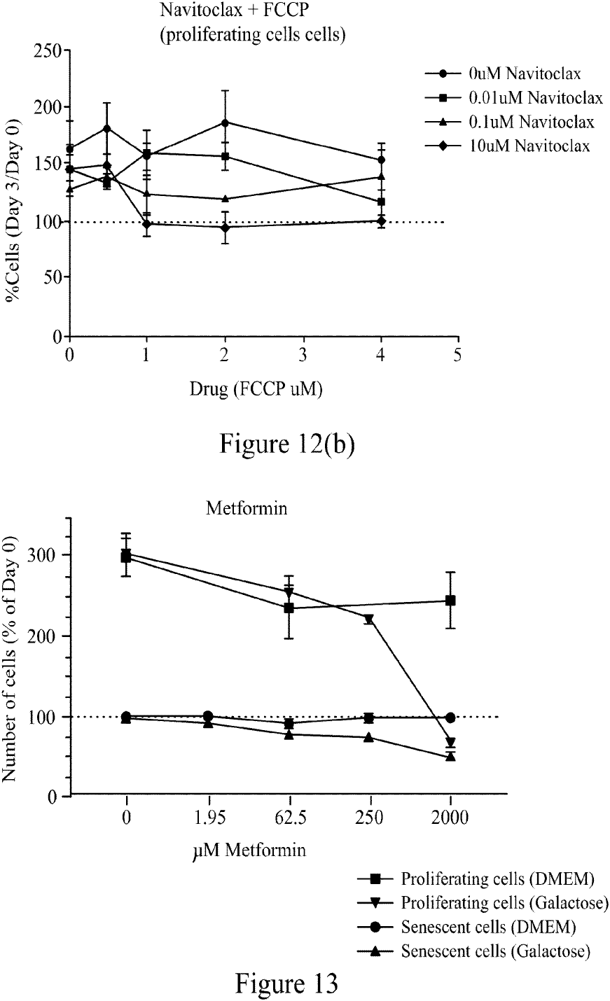

FIGS. 12(a) and (b) illustrate that FCCP increases the senolytic effect of navitoclax, increasing efficacy at lower concentrations in senescent cells and proliferating cells respectively; and

FIG. 13 illustrates that metformin displays senolytic activity in galactose media where glycolysis is suppressed.

### EXPERIMENTAL

### Example 1

### Senescence-Specific Assay

We developed a senescence-specific assay, employing automated counting of differentially labelled senescent and non-senescent cells in co-culture, before and after 72h incubation with each molecule or molecule combination.

Human Dermal Fibroblasts (HDFs) expressing pSLIEW (Green), or mCherry (Red) are used in the assay. The cells expressing pSLIEW (Green) are subjected to 20 Gy X-irradiation to induce senescence. After 10 days, when the cells are deemed to have senesced, the cells expressing mCherry (Red) without prior irradiation hence ‘young’, are added to the culture to create the environment in which both young and senesced cells are co-cultured.

The test molecule is added to the co-culture, and the number of green and red cells are counted before and after 72 h incubation. A selective senolytic would be expected to diminish the proportion of green-expressing senescent cells, while leaving the number of red-expressing young cells unchanged or increased due to replication during the incubation period.

The experiments were carried out in 96-well plates, and the cells were imaged using fluorescent microscopy. The images were analysed with ICY software for automated cell counting, and both numbers of labelled cells and their other visible characteristics such as size were recorded.

### Example 2

### Senolytic Activity of Naturally Occurring Agents

The senescence-specific assay of Example 1 is used to show that the derivatives of the active ingredients quercetin (rutin), apigenin (parsley, parsley extract, chamomile and chamomile extract), EGCG (green tea and green tea extract), honokiol, magnolol (magnolia bark and magnolia bark extract), resveratrol (red grape extract and polygonum cuspidatum root extract) show senolytic activity themselves (either used in isolation or combination).

It will be understood that in this and other later example, alternative senescence-specific assays known to the person skilled in the art may suitably be used. One example of such an alternative senescence-specific assay is described in WO 2018/215795.

### Example 3

### Synergy With Honokiol and Magnolol Combined With Other Senolytic Agents

The senescence-specific assay of Example 1 is used to show that honokiol and magnolol are synergistic with senolytic agents and increase the activity/potency of other known senolytic agents.

### Example 4

### Synergy With Mitochondrial Uncouplers Combined With Other Senolytic Agents.

The senescence-specific assay of Example 1 is used to show that mitochondrial uncouplers are synergistic when combined with other senolytic agents (not restricted to a BCL-2/X MOA).

1-30. (canceled)

31. A composition comprising an amount of an agent effective for the treatment or alleviation of a senescence-associated disease, disorder or effect, wherein the agent is selected from one or more of A42548, A425619, alpha lipoic acid, apigenin, Artemisinin, BML-288, BTBHQ, EGCG, fluphenazine dihydrochloride, honokiol, LE300, magnolol, niclosamide, nicotinamide, nordihydroguaiaretic acid, quercetin, resveratrol and a mitochondrial uncoupler.

32. A composition according to claim 31 comprising an amount of an agent effective for mitigation, alleviation or improvement of the senescence-associated effects of ageing, wherein the agent is selected from one or more of A42548, alpha lipoic acid, apigenin, Artemisinin, BML-288, BTBHQ, EGCG, fluphenazine dihydrochloride, honokiol, LE300, magnolol, niclosamide, nicotinamide, quercetin, resveratrol and a mitochondrial uncoupler.

33-40. (canceled)

41. A composition according to claim 31 wherein the agent is fluphenazine dihydrochloride.

42-44. (canceled)

45. A composition according to claim 31 wherein the agent is niclosamide.

46-48. (canceled)

49. A composition according to claim 31 wherein the agent is a mitochondrial uncoupler.

50. A composition according to claim 31 wherein the composition comprises a combination of two or more of A42548, A425619, alpha lipoic acid, apigenin, Artemisinin, BML-288, BTBHQ, EGCG, fluphenazine dihydrochloride, honokiol, LE300, magnolol, niclosamide, nicotinamide, nitazoxanide, nordihydroguaiaretic acid, quercetin and resveratrol.

51. (canceled)

52. A composition according to claim 31 wherein the composition comprises a combination of resveratrol and EGCG.

53. A composition according to claim 31 wherein the composition comprises a combination of resveratrol, EGCG and quercetin.

54. A composition according to claim 31 wherein the agent is a combination comprising magnolol and honokiol.

55. A composition according to claim 31 wherein the agent is a combination comprising honokiol, magnolol and niclosamide.

56. A composition according to claim 31 wherein the agent is a combination comprising honokiol, magnolol and nitazoxanide.

57. A composition according to claim 31 wherein the composition comprises niclosamide in combination with one or more of resveratrol, alpha-lipoic acid (ALA), niacinamide (also known as nicotinamide (NAM)), epigallocatechin gallate (EGCG) and fluphenazine dihydrochloride.

58. A composition according to claim 31 wherein the composition comprises the combination ABFGH.

59. A composition according to claims 31 which includes a BCL2/X inhibitor.

60. A composition according to claim 59 wherein the BCL2/X inhibitor comprises one or more of navitoclax and A1331852.

61. A composition according to claim 31 wherein the composition includes metformin.

62-65. (canceled)

66. A composition comprising an amount of an agent effective for the treatment or alleviation of a senescence-associated disease, disorder or effect, wherein the agent is a mitochondrial uncoupler.

67. A composition comprising a mitochondrial uncoupler in association with an amount of an agent effective for the treatment or alleviation of a senescence-associated disease, disorder or effect.

68. A composition according to claim 66 wherein the mitochondrial uncoupler is selected from one or more of Carbonyl cyanide-p-trifluoromethoxyphenylhydrazone (FCCP), Carbonyl cyanide m-chlorophenyl hydrazone (CCCP), N5,N6-bis(2-Fluorophenyl)[1,2,5]oxadiazolo[3,4-b]pyrazine-5,6-diamine (BAM 15) and 2,4-Dinitrophenol (DNP).

69. (canceled)

70. A composition according to claim 31 wherein the senescence-associated effects of ageing include age related skin conditions, skin conditions related to sun exposure, skin conditions related to pollution exposure, skin conditions related to oxidative stress, and skin conditions related to lifestyle choices, such as diet, alcohol and/or smoking.

71. A composition according to claim 70 wherein the senescence-associated effects of ageing include skin conditions related to inflammatory skin disorders and skin conditions related autoimmune disease skin disorders.

72. A composition according to claim 31 wherein the senescence-associated effects of ageing include increased frailty, loss of resilience, loss of muscle strength, loss of muscle endurance, loss of energy, loss of cognitive sharpness, loss of memory, etc.

73. A composition according to claim 31 wherein the senescence-associated effects of ageing include atherosclerosis and cardiovascular disease, cancer, arthritis, cataracts, osteoporosis, type 2 diabetes, hypertension, Alzheimer’s disease, glomerulosclerosis/decline in renal function.

74-91. (canceled)

92. A method of mitigation, alleviation or improvement of the senescence-associated effects of ageing in a host, said method comprising the administration of an effective amount of a composition comprising one or more of A42548, A425619, alpha lipoic acid (B), apigenin (C), Artemisinin, BML-288, BTBHQ, EGCG (H), fluphenazine dihydrochloride, honokiol, LE300, magnolol, niclosamide, nicotinamide (F), nordihydroguaiaretic acid, quercetin (G), resveratrol (A) and a mitochondrial uncoupler.

93-111. (canceled)

112. A method according to claim 92 wherein the agent is a mitochondrial uncoupler.

113-116. (canceled)

117. A method according to claim 92 wherein the composition comprises a combination of resveratrol, EGCG and quercetin.

118. A method according to claim 92 wherein the composition comprises a combination of magnolol and honokiol.

119. (canceled)

120. A method according to claim 92 wherein the agent is a combination comprising honokiol, magnolol and nitazoxanide.

121. A method according to claim 92 wherein the composition comprises niclosamide in combination with one or more of resveratrol, alpha-lipoic acid (ALA), niacinamide (also known as nicotinamide (NAM)), epigallocatechin gallate (EGCG) and fluphenazine dihydrochloride.

122. A method according to claim 92 wherein the composition comprises the combination ABFGH.

123. A method according to claim 92 which includes a BCL2/X inhibitor.

124. A method according to claim 123 wherein the BCL2/X inhibitor comprises one or more of navitoclax and A1331852.

125-134. (canceled)

135. A method according to claim 92 wherein the senescence-associated effects of ageing include skin conditions related to inflammatory skin disorders and skin conditions related autoimmune disease skin disorders.

136. A method according to claim 92 wherein the senescence-associated effects of ageing include increased frailty, loss of resilience, loss of muscle strength, loss of muscle endurance, loss of energy, loss of cognitive sharpness, loss of memory, etc.

137. A method according to claim 92 wherein the senescence-associated effects of ageing include atherosclerosis and cardiovascular disease, cancer, arthritis, cataracts, osteoporosis, type 2 diabetes, hypertension, Alzheimer’s disease, glomerulosclerosis/decline in renal function.

138. A method according to claim 135 wherein the senescence-associated effects of ageing is an age related skin condition.

139-142. (canceled)

143. A method according to claim 138 wherein the skin condition includes one or more of acne, asteatotic eczema, atopic dermatitis, contact dermatitis, discoid eczema, eczematous drug eruptions, erythema multiforme, erythroderma, gravitational/varicose eczema, hand eczema, keratosis lichenoides chronica, lichen nitidus, lichen planus, lichen simplex, lichen striatus, mycosis fungoides, pityriasis lichenoides, psoriasis, seborrheic dermatitis, Stevens-Johnson Syndrome, toxic epidermal necrolysis and vasculitis.

144-157. (canceled)

158. A composition according to claim 31 wherein the agent is selected from one or more of A425619 (recombinant human protein), A42548 (recombinant human protein), artemisinin, BML288, BTBHQ, fluphenazine dihydrochloride, honokiol, LE300, magnolol, niclosamide, niacinamide and nordihydroguaiaretic acid.

159. A method according to claim 92 wherein the agent is selected from one or more of A425619 (recombinant human protein), A42548 (recombinant human protein), artemisinin, BML288, BTBHQ, fluphenazine dihydrochloride, honokiol, LE300, magnolol, niclosamide, niacinamide and nordihydroguaiaretic acid.

160. A method according to claim 137 wherein the senescence-associated effects of ageing osteoarthritis.
